(19) United States
(12) Patent Application Publication
(10) Pub. No.: US 2019/0362957 A1
(43) Pub. Date: Nov. 28, 2019

(54) A DEVICE AND A METHOD FOR SCREENING OF SMALL TO MID SIZE LUGGAGE FOR TRACES OF ILLICIT SUBSTANCES

(71) Applicant: KARSA OY, Helsinki (FI)

(72) Inventors: Jani HAKALA, Vantaa (FI); Verner HEMMILÄ, Ohkola (FI); Hans-Jurg JOST, Vantaa (FI); Heikki JUNNINEN, Helsinki (FI); Juha KANGASLUOMA, Helsinki (FI); Jyri MIKKILÄ, Helsinki (FI); Aleksei SHCHERBININ, Helsinki (FI); Mikko SIPILÄ, Helsinki (FI)

(21) Appl. No.: 16/333,110

(22) PCT Filed: Sep. 10, 2017
(86) PCT No.: PCT/FI2017/050642

§ 371 Date: Mar. 13, 2019

(30) Foreign Application Priority Data
Sep. 19, 2016 (FI) 20165702
May 23, 2017 (FI) 20175460

(51) Int. Cl. H01J 49/14; G01N 1/40; G01N 1/22; G01N 33/22

(52) U.S. Cl. CPC H01J 49/145; G01N 1/40; G01N 2001/024; G01N 33/227; G01N 1/22

(57) ABSTRACT

Disclosed is a system for screening of traces of illicit substances that may be harmful, such as explosives, radioactive substances, toxics or drugs for example, from very tiny trace concentrations to be detected by way of mass spectrometry being applied to the detached and pre-concentrated particulate matter by the system. Also disclosed is a method in accordance of the system, an arrangement or device as a system element of the system, and a software code on computer readable medium, to control the system and/or acquire data from the system.

In very general level the invention relates to detection of very small amounts of matter, but more specifically to detection of such matter that is detached from the carrying matrix, but even more specifically to such detection as disclosed, but additionally such matter that is considered illicitly hazardous somehow. The technical field of such a detection device is more specifically defined by the preamble part of an independent device claim directed to such a device. Such a device is embodied to belong as a system element to a system in the same technical field as more specifically defined by the preamble part of an independent system claim directed to such a system. A detection method using the embodied system element in the same field of the system and the system element belongs to the more specifically to same technical field. The invention concerns also software module that is used in the detection, but also such software module that is used in the automated control of the system element operations as a system.

### BACKGROUND

Illicit substances (IS) are considered such because of the hazardous properties of such substances. Into such class of ISs belong such substances as toxics and explosives, exemplified as super-poisons, nerve gases, narcotics, drugs, explosives, and radioactive substances, for example. The illicit nature via the hazard to any life forms may be sufficiently clear without any further disclosure of these kinds of substances.

However, ISs as such as handled properly according to the international agreements to follow safety regulations in supervised conditions are not problems. But the smuggling, use related to sinister purposes and terrorism as well as careless dealing with such subjects, especially in secrecy, makes a considerable problem, in world wide.

For illegal use such substances are often hided, when stored and/or transported, even among large crowds, ignorantly about the risk such substances may cause if released, in purpose and/or accidentally. Some of these substances have so low vapor pressure in the normal conditions that they cannot be traced immediately, but a very long analysis period is needed, especially for Extremely Low Vapor pressure having Organic Compounds, ELVOCs, of which some are suitable for illicit purposes.

In warehouses such slow detection operation may be acceptable, but for example in airports, harbors or public transport stations, where hundreds of passengers carry their luggage in and out in a very short time, it is not acceptable, even the gained safety and even the terrorists and/or drugs smugglers were revealed, for example. One weakness of the known systems relays on that if the traces were collected for long, especially in case of ELVOCs the amounts of the traces are so small that there are many false alarms triggered erroneously because of the poor statistics.

Thus, it is an aspect of the invention to bring up such a system that can detect these illicit substances or alike rapidly, reliably, and accurately for reporting the findings accordingly for further action.

Such aspect if achieved by the system that is claimed in the independent claim, system element and the method to operate such a system according to the characterizing part of respective independent claims directed to thereof.

System according to the invention to detect Traces of Illicit Substances, TOIS is characterized by that what is said in the characterizing part of an independent claim directed to said system.

System element of such an aforementioned system to detect Traces of Illicit Substances, TOIS, according to the invention is characterized by that what is said in the characterizing part of an independent claim directed to said system element.

Method to detect Traces of Illicit Substances, TOIS according to the invention is characterized by that what is said in the characterizing part of an independent claim directed to said method.

The invention concerns also software module in accordance of the aspect, to be used in the detection, but also such software module that is used in the automated control of the system element operations as a system.

Other embodiments and their examples are indicated in the dependent claims.

Embodiments of the invention are now explained in a further detail by referring to the following figures (FIG), in which

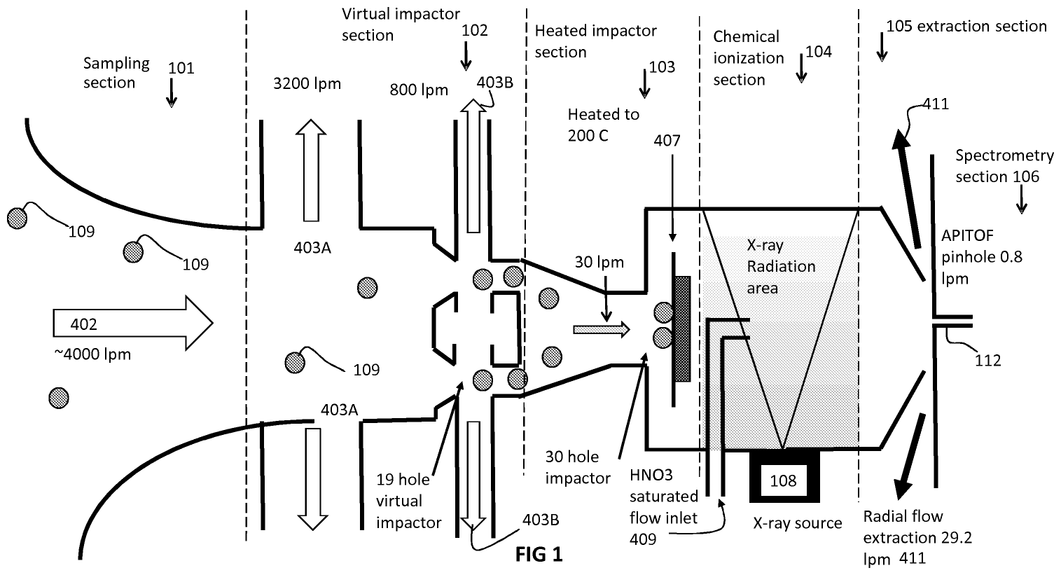

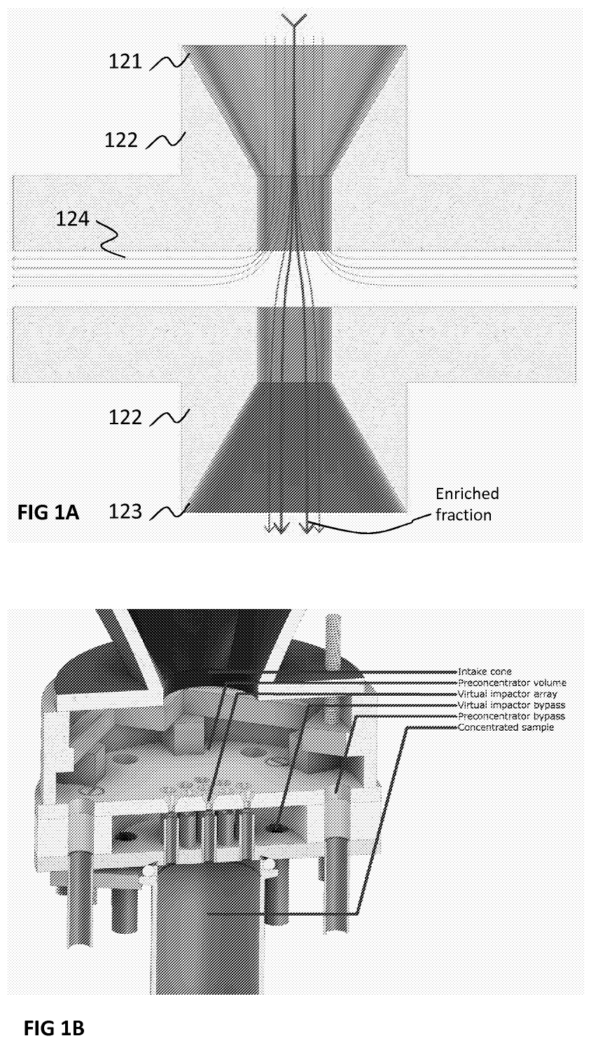

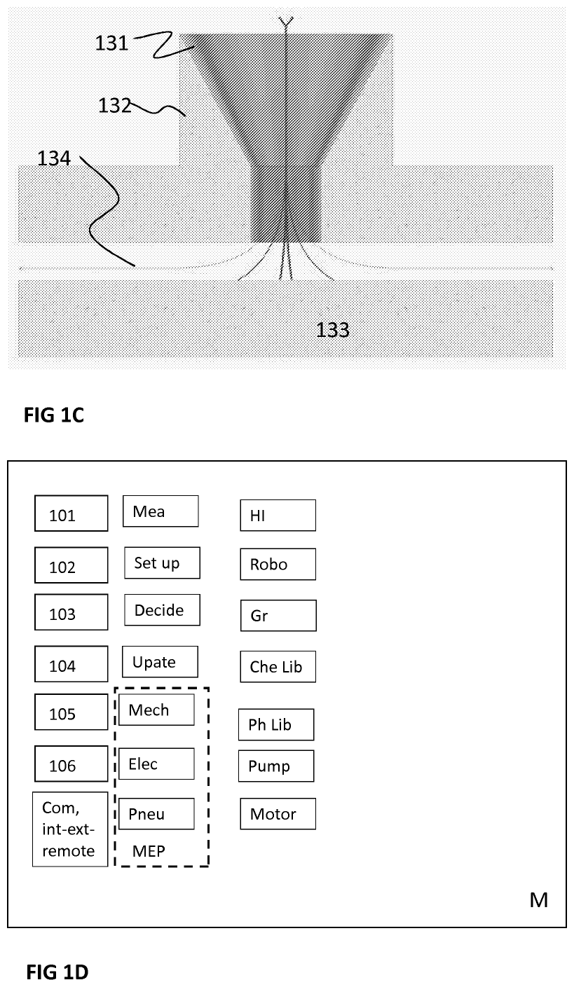

FIG. 1 illustrates embodiments of the invention as a schematic view to the system comprising system sections as modules,

FIG. 1A illustrates operating principle of an virtual impactor as such,

FIG. 1B illustrates two virtual impactors in series as a system element of the embodiment of the invention

FIG. 1C illustrates impactor operating principle as such,

FIG. 1D illustrates system software module, from a general view of operations of the system,

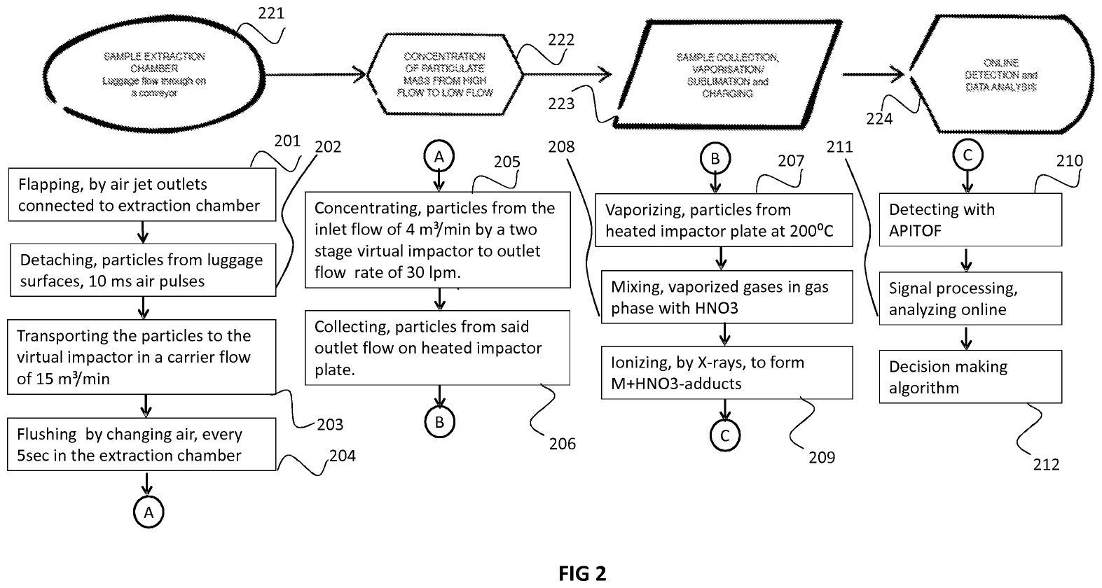

FIG. 2 illustrates operation of the system via embodiments directed to method,

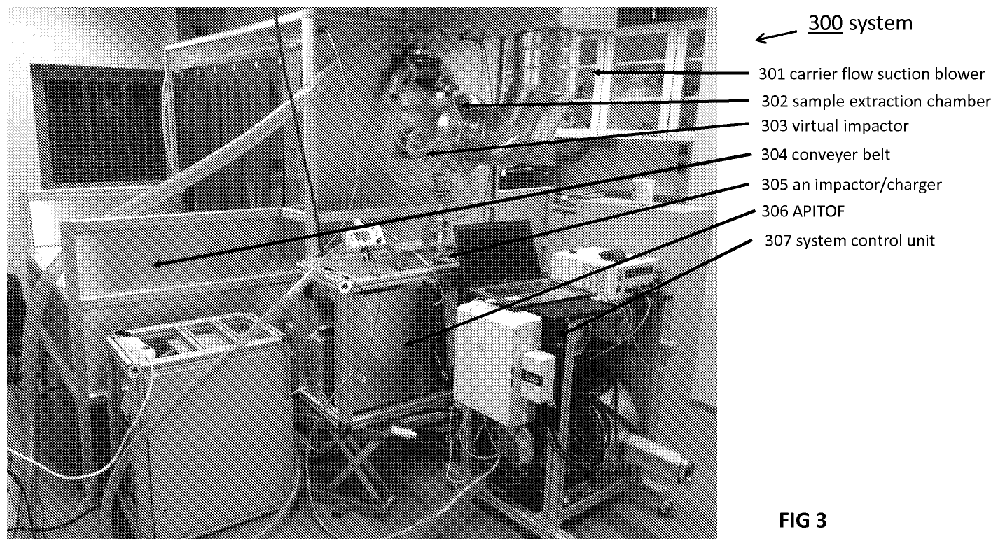

FIG. 3 illustrates implementation of the embodied system, via a photograph,

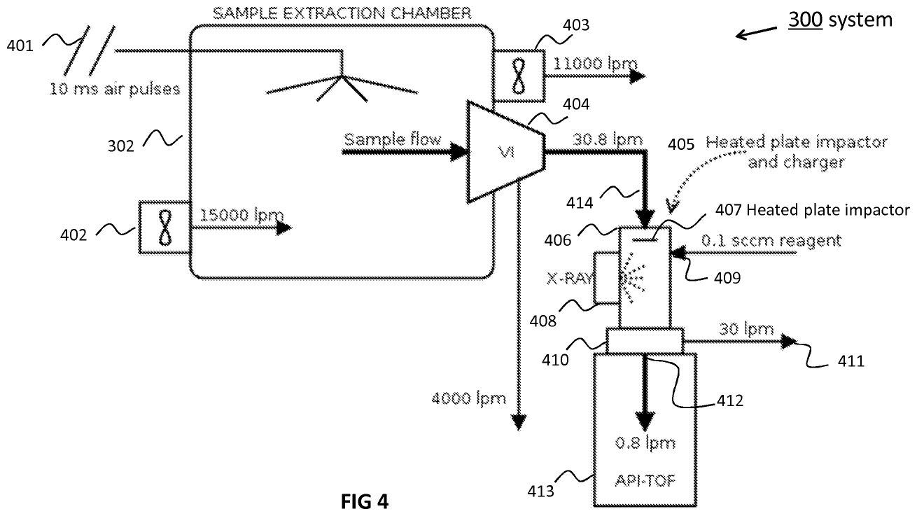

FIG. 4 illustrates embodiment of the system via a schematic view,

FIG. 5 illustrates an embodiment of an ensemble of system elements as a cross sectional view

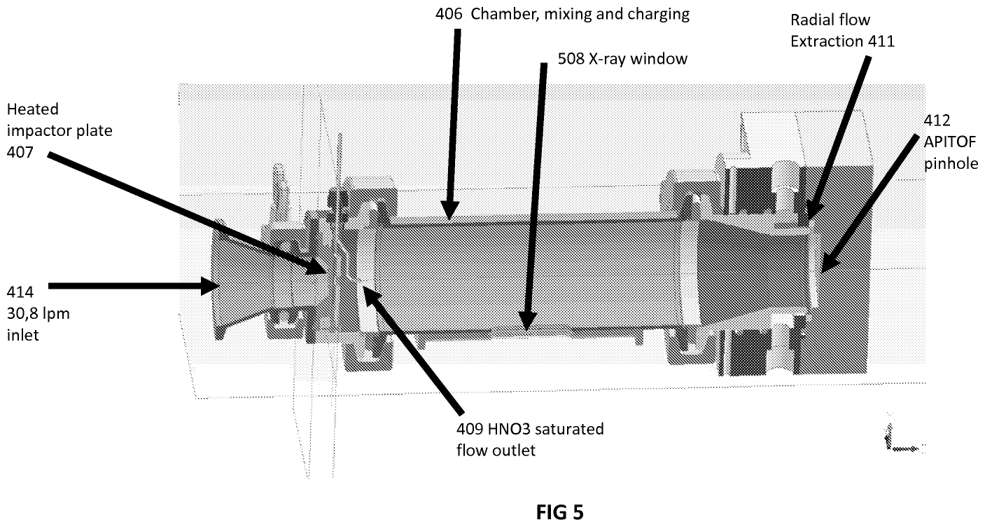

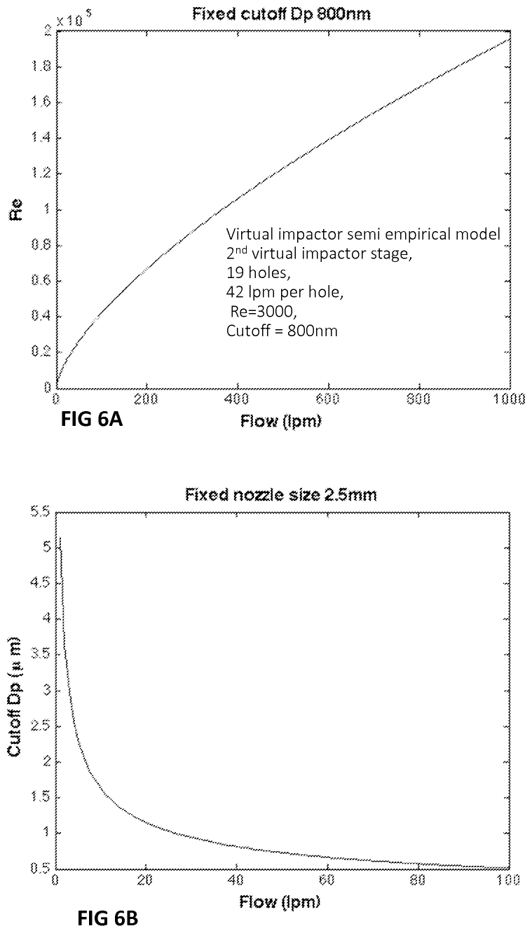

FIGS. 6A, 6B illustrate virtual impactor characteristics curved for a semi empirical model of 2^{nd} virtual impactor, cutoff and Reynolds number Re dependencies are illustrated,

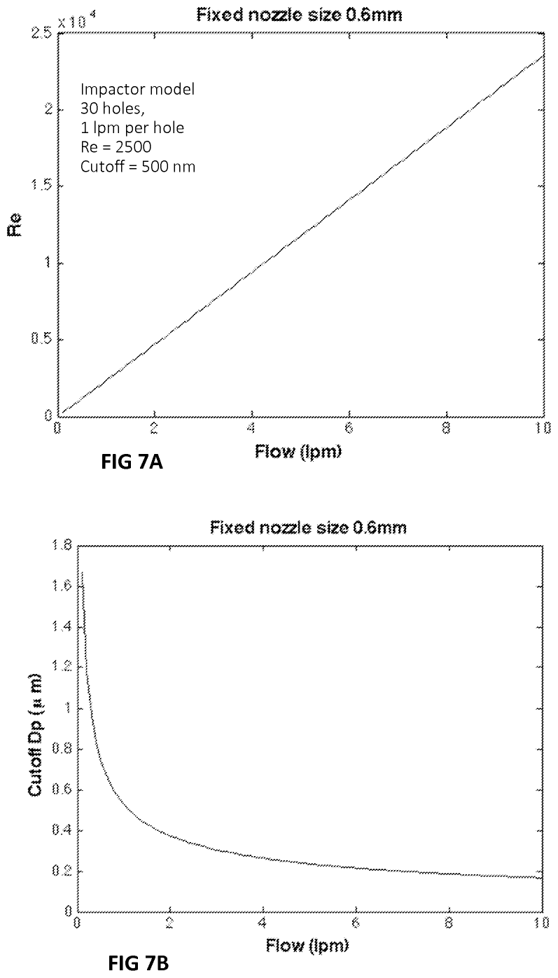

FIGS. 7A, 7B illustrate impactor characteristics curved for an impactor model, cutoff and Reynolds number Re dependencies are illustrated,

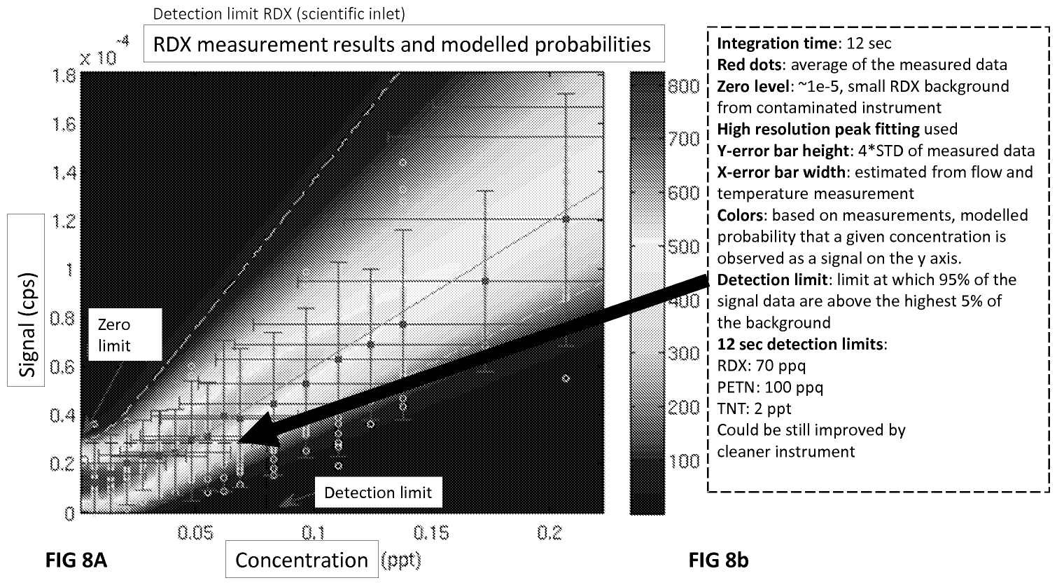

FIGS. 8A, 8B illustrate graphics- and text-fields, respectively, on a virtual computer screen for an RDX measurement containing also simulation data example,

FIG. 9 illustrate graphics- and text-fields, on a virtual computer screen for an ensemble of explosive sample tests results, and

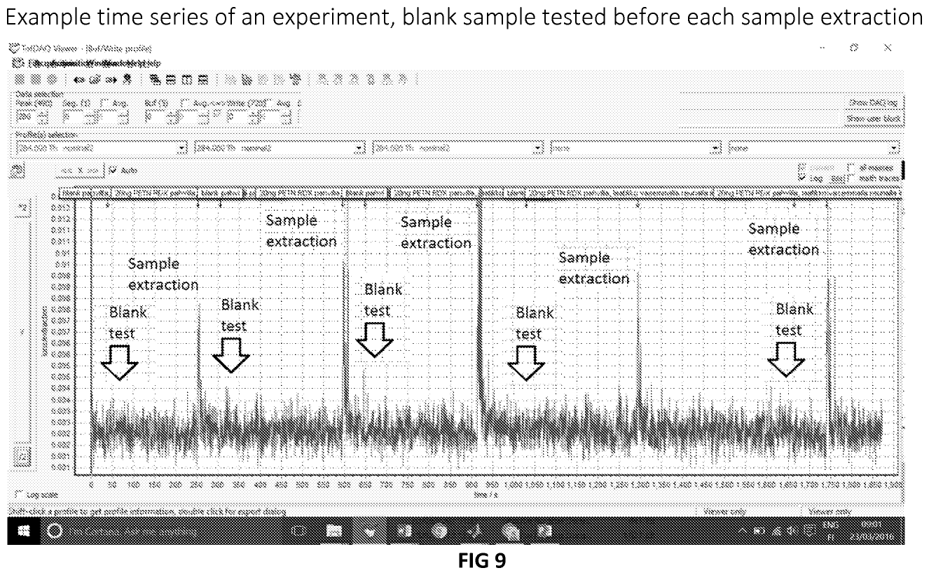

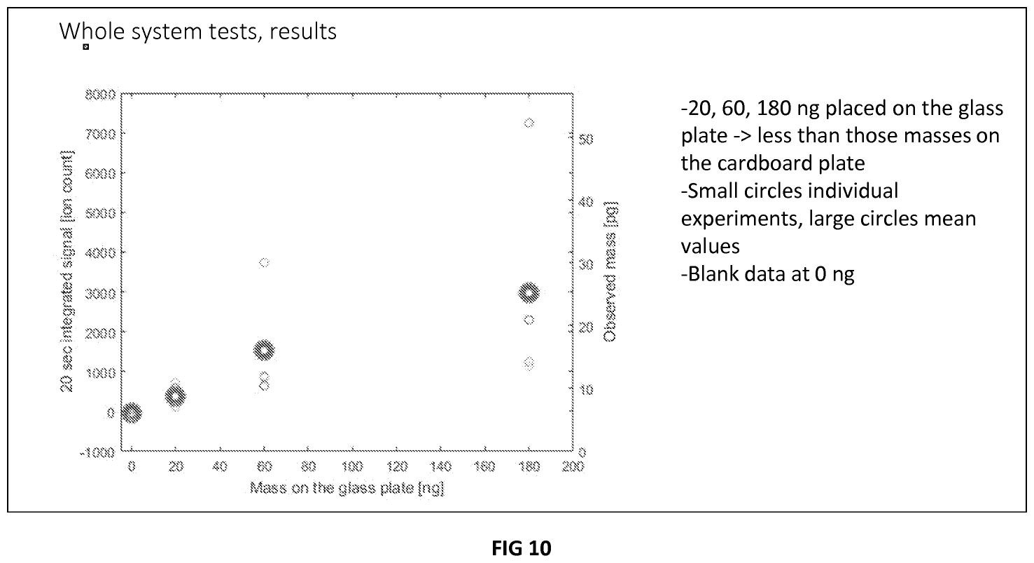

FIGS. 10-19 illustrate testing results of the embodied system on a screen.

Embodiments of the invention are combinable in suitable part. Same reference numerals are used for the same kind of objects. The objects do not necessarily need to be identical, except where especially otherwise indicated in respect of the identical nature and/or differing use of the reference number in another figure (FIG) and/or text part.

The indicated values in the drawings are only examples which do not limit the scope of the invention only to the shown exemplified embodiments.

FIG. 1 illustrates schematically a system according to an embodiment of the invention for an implementation of such a system as a device. The device may be embodied inside into one cover in suitable part to form the device form the system elements, but they as system modules may be mounted in separate for a functional unit in suitable part, so that they together form the functional arrangement from the system elements. Embodiments that form a functional arrangement can be regarded easier to maintain and repair or update, if such a procedure were needed. However, a device may be more compact and preferred for mobile units. According to an embodiment variant, even the extracting chamber to detach the particles can be integrated to the device, the electronics, pneumatics and mechanical setup being thus incorporated to the same unit in a suitably sized carriage.

The dashed vertical lines illustrate exemplified sections that are comprised in the system as system elements of the system in an embodied to form a device or an arrangement. As the vertical dashed lines indicate an example, it is not intended to limit the scope of the embodiments only to the shown example. Thus, a skilled person in the art knows from the embodiments that the functional modules can be integrated pair wise, or triplet-wise, to form modules according to embodiment variants having same functionality for the system, but built up from more integrated modules than such a setup that is made from separate items. A system according to an embodiment comprises in the example of the FIG. 1 (FIG. 1) a sampling section 101, virtual impactor section 102, heated impactor section 103, chemical ionization section 104, extraction section 105 and spectrometry section 106. The computer section is not indicated in the FIG. 1, neither the interfaces to the spectrometer, which can be implemented according to the suitable standard parts, not the automation related actuators, which can be used as in normal industrial automation to set timings for conveyors, gates, and manipulators, to guide pneumatic air jet system part and match the timings for the operation in whole.

However, in an embodiment of the system there can be also computerized part that comprises the routines, drivers and operational data to the indicated sections above to be operated in a modular way to collect data and/or send commands for control to each these modules formed from the sections. The computer module can comprise also the data libraries about the molecules and/or their mobilities in suitable part for the mass spectrometer.

According to an embodiment variant the computer for the spectrometer operation control and the related analysis can be remotely operable via internet or another communications network. For such embodiment there can be terminal device to provide the necessary connections for the data transfer and commands between the actuators and the remote computer.

The computer module can comprise also a data section for tables, and databases, for the operational parameters, timings, signal processing and filtration parameters, but also connections and the protocols to be obeyed in the communication with the modules, as well as specifications for parameters to be used within the external terminals as well as other devices, if needed for assistance for the infrastructure to maintain the operation of the system and/or its parts in the system.

The computer module comprises an operating interface for human operator for the settings and controlling, but also such drivers and means that can be used for guiding a human operator in doing sampling in a vessel or warehouse. However, having a human interface, the system can be embodied to operate autonomously according to the controlling parameters and/or the set up settings. According to an embodiment of the invention, the system produced data is facilitated to get outputted via an interface to another computer or terminal in an extraneous system.

At the beginning when following the sample to be taken, it is first detached from the surface by air jet pulses, and/or a flow, to get particulate matter airborne and into the sampling flow. According to an embodiment of the invention the sampling flow is considerably high, i.e. about 4 m³/minute, but it can be even higher for parallelly cascaded virtual impactor modules that comprise such virtual impactors as the module 103. This way, from the same volume, by using parallel sampling lines for the same sample of detached particles, more particulate matter can be released, and in an embodiment variant the concentrated samples by the virtual impactor can be put together, for achieving even further lower detection limit.

In FIG. 1 the sampled airborne particles 109 are carried to the virtual impactor module 102, which in the example is implemented by two virtual impactors in series. In FIG. 1C there is illustrated an operation principle of an ordinary impactor. The particle sample to be collected by the impactor is entering to the impactor nozzle area 131, which is made conical in this example into the surrounding material, forming a nozzle wall 132. The particles are brought to the impactor airborne in a flow, which is demonstrated in the figure by the vertical lines. When the flow line, along which a particle is travelling airborne in the flow, makes a sudden turn at the plate 133, the curve-linear motion of the gases guides the gases aside so to get escaped into the outlet channel 134 and out of the impaction area just below at the flow entry to the volume. Because of the inertia of particles in the flow, they are not able to follow the gas molecule path with the gas molecules, and the heavier particles just hit the plate 133. It is the inertia on which the impactor's operation is based, and some of the smaller particles may escape the sudden turn to the direction of the carrier medium gas, if they are sufficiently small in mass size. The cut size that is used as a characteristic measure of an arbitrary impactor stage is often meant as a 50% cut size, at which particle size half of the particles (having this mass at the very size) are collected, the larger ones more effectively, the small ones less effectively. The cut size is defined by the nozzle to plate diameter, nozzle geometry and the flow rate and its turbulence (Reynolds number Re). Because of the inertia, increasing the flow velocity also influence to the cut size so that smaller particles can be collected.

A virtual impactor of FIG. 1A differs from an impactor in that, there is no such collection plate to get the particles onto a pile below the nozzle, at the plate, but instead a hole fitting to the geometry so that it guides the particles through the plate that in impactor were about to pile at the plate under the nozzle the particles. The term under or below are not limiting the scope of the embodiment by any means, as they are only used to denote to the normal position of the paper in western reading and writing system when one is about to read or write.

Thus, in a single virtual impactor stage as embodied herein in FIG. 1A, there is a nozzle 121, the nozzle wall material 122, and the tunnel 124 for the extra gases for their escape, that are left over from the stage, in an embodiment to be used in pre-concentration as particle lean.

In the example, the down part appears to be as it were mirrored in respect to the tunnel 124, but it is not necessarily so, as the flow with the pre-concentrated particles is typically smaller than the flow at the entry to the stage.

The outlet cone can be designed and machined so that it gives space to the gases to swell and consequently so to accelerate the particle velocity. Accordingly when the particles in the flow after the first stage are carried to the next virtual impactor stage, the deviation of the particle jet is getting so smaller, and the operation of the next stage is made so easier to get a better representative sample. According to an embodiment variant the outlet trajectory cone is made wider than the inlet cone.

An exemplified structure of a two stage virtual impactor is shown in FIG. 1B, which shows the intake cone, leading to the sample extracting chamber (via suitable tubing for example), pre-concentration volume, inside the virtual impactor serial cascade, at the interface between the stages of the virtual impactor. Virtual impactor array is indicated, for a smaller inflow, in which the sampled particles are concentrated and the gaseous species as such are directed to the outflow, as for each stage. The side tunnels bypass the extra, particle lean gases out form the second virtual impactor stage too. The flow is divided in both stages to several nozzles to form jets accordingly. Concentrated sample continues from the second stage of the virtual impactor for the next module.

Although a structural example of the virtual impactor module is shown, a skilled person in the art knows from the embodiments of the invention that the virtual impactor stages can be cascaded in serial way, similar to the shown example of FIG. 1B, but also in parallel to divide a larger volume of sample to separate sub-sample lines.

FIG. 1 also shows exemplified values for virtual impactor outflow represented by the arrows 403A, 403B, for the two embodied virtual impactor stages so that the flows are for the first stage 3200 lpm, and for the second stage 800 lpm. The residual sample flow of 30 lpm is also taken from the same input origin, and is included to the approximately 4000 lpm sample flow 402.

In FIG. 1 the final concentrated sample is carried in the 30 lpm flow to the heated impactor section 103 comprising at least a collecting unit. The heated impactor as such operates as a normal particle collecting inertial impactor, but however, the heating of a collecting plate 407 in the embodiments of the invention makes the volatile substances on the collection plate to get vaporized, when the plate is heated, by electrical means, which may be utilizing resistive heating elements, and/or eddy current based magnetic field based heating elements in the system. According to an embodiment of the invention the impactor can have even further stages, (one or two, even double stages as an ensemble of embodiment variants), to reduce the bouncing that as such is considered as a problem in impactor-made sampling science. However, also cyclone designed sampling unit can be embodied in the system to be used instead of an impactor, or in an embodiment variant with more than one collecting units, parallel to an impactor, to be heated in suitable part.

The collection is expected to occur same ways as in normal impactor operation, with the environmental conditions applied as they are for the elevated temperature, collecting also the explosives containing particles, that when occurring in the dusts, attach to the mineral particles etc, but if collected as such, or as attached to other particles, evaporate when the temperature is suitably selected. In one embodiment, the plate could be heated to 200° C., so RDX for example is supposed to get vaporized into the gas phase. However, the temperature is not limited to the shown example value of 200° C.

According to an embodiment, the dusts may pile to the heated impactor plate 407, and little by little may be blocking the impactor's passage. So there might be a need to exchange the plate as such.

According to an embodiment of the invention the plate can be operable as one in an ensemble of similar kind of plates, which are removable and replaceable. The exchange maneuver can be made manually in an embodiment in suitable part, but can be automated for an automated embodiment so that when measuring pressure over the impactor stage, the measured pressure over the impactor stage is about to get too high, i.e. above a pre-determined, impactor stage bound geometry designed threshold level, which is set into the data base of process parameters for the automated system operations, and so to be supervised, and to be used as an initiative to generate an exchange signal to the actuator to change the full plate to a new one, when the threshold level is reached or exceeded. According to an embodiment exchange signal can be generated to have the stage exchanged for other automation, and/or operator detected reasons. For example if a contamination were suspected that would harm the system or operator or other human beings, detected by the mass spectrometer or otherwise, the stage could be exchanged. Also other functionality can be commanded in such control, for example such as shut down the system and/or to close inside the entire luggage hermetically, if the luggage were associated to the hazard so observed.

According to an embodiment of the invention the collecting substrate of the collecting unit, in an impactor a plate, can be used automatic cleaning. However, according to an embodiment variant of the invention, the cleaning can be accomplished by using at least one of the following in suitable part: flow, reverse flow, pulsating flow, solvents, such as bath, mechanical and thermal agitation as such.

According to an embodiment of the invention the new plate, as the old one, is one of an ensemble of such in a chain that can be a linear chain or revolver type of chain. They may be also in a pile, from which one is taken from the top or bottom to be used in the collection, as an optional embodiment to a ribbon type adjacent feeding system. However, when a new one is in duty, the old one can be cleaned for next use, or, it can be archived for a closer and more thoroughly inspection. Sometimes the time scales may be sufficient to permit a longer analysis of the plate as such for example, so that the substances can be detected. If for example a plate is analyzed for an hour sampling time, if there were a reason to do so, it is possible to have an alarm or warning about anomalous content sampled from a batch of luggage or parcels.

According to an embodiment variant, the collection of the sample to the heated plate can be made on a metal foil for example, that is rolled from one roll to another. This way such rolls can be analyzed for the substances thereon, afterwards for example by gamma- and/or X-ray spectrometer, for the species on the rolls, when a used roll is finally exchanged to another and while a new one being in the operation in duty. It is also possible to archive interesting samples, especially those on the rolls for further considerations, for example on chemical analysis basis.

In the chemical ionization section 104 the entering flow is irradiated by a soft X-rays produced by an X-ray source 108. According to an embodiment of the invention the X-rays are produced by an X-ray tube, for example, whose operating voltage is below 100 kV, preferably below 50 kV, but even more preferably less than 25 kV. According to an embodiment the operating voltage is however, above 1 kV, preferably over 1.5 kV, more preferably over 1.8 kV, but even more preferably over 2 kV, according to a variant of the embodiment the voltage is set between 3-5 kV to have the X-ray energy to suitable range.

According to an optional embodiment, the charging can be made also by a corona discharge based ionization, in suitable part, even in addition to the X-rays or UV in an ensemble of embodiment variants, the ionization so being used to produce ions into the volume. The electric field can be then between 0.8 and 8 kV/cm. The charging/ionization geometries can be modified from the well-known electrostatic precipitator geometries as such to comprise wire-plate, needle-to-plate or plate-to-plate geometries in respective optional geometries. In embodiments that involve additional radiation, the direction of radiation is chose so that the structures do not screen unnecessarily the radiation. However, radiation can be also filtered by suitable filters to produce softer X-rays, which may be useful in such embodiments that integrate to the conveyor belt inspection devices and systems using X-rays in luggage detection, but the radiation is more energetic than desired for the ionizing purposes of the embodiments.

According to a further optional embodiment of the invention, for detection of suitable substances having illicit nature, the charging can be made in suitable part by UV-source based radiation, such as an excimer tube or lamp, for example.

For the combination of the vaporized matter molecules form the heated impactor plate, as a reagent of HNO₃ in a saturated reagent flow 409 is introduced (FIG. 4) to the chemical ionization section. According to an embodiment of the invention the chemical ionization module can comprise for the HNO₃ saturated reagent flow source and the related means. According to an embodiment, also other reagents can be used, which match, if needed better to the chemistry of the detectable traces. According to an embodiment also combinations of reagents can be used for the adduct production.

According to an embodiment of the invention, the 30 lpm flow (value embodied, but used as a non-limiting example value to only that) is divided to the radial extracted flow to be directed out of the system, which however, according to an embodiment variant can be treated by a filter, electrostatic precipitator and/or cyclone to collect a cumulative sample. This kind of cumulative sampling, for the sort of exhaust-type gases to get removed, can be made also in option or additionally to the flows 403A and 403B individually or as combined. In case of electrostatic precipitator (ESP) in use for the cumulative sampling of the potentially escaped particulate matters, a laminar flow type ESP is preferred, because of the design option of 100% collection for the cumulative sample, to be further analyzed.

With the cumulative sampling, it is possible to estimate the yield that enters to the mass spectrometer from the released substances. It is also so possible to made a post-analysis of the cumulative sample with a suitable analytical system to analyze the composition and/or the constituents from the collected mass, to reveal such anomalies that could have been possibly considered as normal and treated as such in the analysis. According to an embodiment suitable radiation can be used in the analysis.

According to an embodiment of the invention a reagent may be a dimer, trimer etc. or (HNO₃)ₙNO₃⁻.

According to an embodiment of the invention evaporation, heating and/or X-rays can be used to fragment the substance molecule. According to an embodiment the finger print fragment is detected as an adduction direct ionization product.

For example, if a luggage of a largish passenger group has been screened, nothing as such found, the group being large and the luggage well wiped to be extremely clean, so that the system could have possibly considering these pieces of luggage just as they were normal, but clean, so to be so clean by an accidental way being so clean, the cumulative sampling may find those small residues that could have detached from the well cleaned and wiped luggage at the detachment/extraction phase, as well as the pre-concentration of the system's normal sampling. So the system can get an alarm, for example that RDX was detected, or money printing ink residues were found, or several drugs found for example.

Then, in some cases it is possible to unload the cargo and make a full inspection to the estimated number of luggage, before the departure, or an already departed flight may be called back to the nearest airport to get the passengers evacuated.

At the extraction section 105 the flow is divided, according to an embodiment the sample containing flow is lead to the APITOF leading line in the exemplified 0.8 lpm flow via the flow line 112.

Because of the x-rays and vapors in the medium in the chemical ionization section, the conditions inside are favoring the substances in a gaseous phase, to form combinations of the substances.

The original particles that carry the illicit substances can be detected by sample preparation finally by the APITOF mass spectrometer.

According to an embodiment of the invention, X-rays are used for ionization to produce NO₃⁻ from the HNO₃, which makes an adduct NO₃⁻ molecule, which can be detected with the APITOF. According to an embodiment also direct ionization of substance molecule with X-rays is possible to be get utilized. So, according to an embodiment of the invention, the detectable substances are in gas phase at this stage of the sample analysist path from the extraction at the beginning to the mass-spectrometry at the end.

According to an embodiment of the invention, also other reagents can be used, other than merely HNO₃. According to an embodiment also I₂, acetone, HCl, O₂, for example, can be used as reagents, in suitable part alone or in combination. According to an embodiment also HNO₃ can be present in a multicomponent reagent combination.

According to an embodiment, the mass spectrometer is tuned so, that it recognizes also isotopes of the substances by their abundances in the sample. This can be achieved so that it is known at what masses the isotopes should be found, and the abundances in nature are known, so by comparing the found isotope masses that co-incident with other masses can so reveal that a luggage may have been a target of manipulating its traces to cause false alarms for example and consequently to degenerate the detection.

FIG. 2 shows an example of an embodiment of the invention directed to a method of detecting online by the embodied system variant illicit substances. What is illicit, it can be updated to the computer module.

The method has phases such as sample extraction 221 in a suitable chamber. In an airport such a chamber can be for example a one designed for the purpose, but optionally also the X-ray tunnel in suitably closed manner could perform the same operation as the chamber, when it is modified according to the embodiments to comprise the air flows for the airborne particle sample transportation to the further system parts. The air jet pulse generating means can be also added, so that the system can flap 201 the luggage flowing through on a conveyor 221. The flapping 221 by pressurized air or another inert gas in the chamber detaches 202 particles from the luggage surfaces.

The pulses of the air jets can be in the scale of 10 ms as its duration, although other durations can be used for different luggage types, which can be recognized from the belt by the automation. According to an embodiment also a continuous air blade can be used, instead of the pulses, or in addition. According to an embodiment variant ensemble also different duration having pulses and/or continuously maintained air blade can be used in operation, with an intermittent sequences, if not embodied as such with constant durations of them. For example wet or moist luggage may need a different type of pulsation for the air to have the particles detached. According to an embodiment, also electrostatic ejection (based on electrostatic influence of the charge applied on luggage) can be used in suitable part, especially if it is clear that there should not be any such content in the luggage that would be damaged. According to an embodiment thermal desorption and/or desorption-electrospray-ionization can be used in supplement or in addition.

The detached particles that become airborne are transported 203 to the virtual impactor module in the carrier flow. In FIG. 2, a flow of 15 m³/min is shown as an example, in which part of the volumetric flow can be used for flushing 204 the extraction chamber. For example, every 5^{th} second change the air to have new atmosphere. The continuation points of the method in further phases are indicated by the letters A, B and C, as encircled for the drawing based reasons only.

At the virtual impactor module, the particulate sample is concentrated 205, from a high mass flow to a low mass flow 222. According to an example of embodiment, the inlet flow of 4000 lpm is reduced to such a low mass flow as 30 lpm, by a two stage virtual impactor (FIG. 1B).

The particles concentrated in the two stage virtual impactor, are collected 206 onto a heated plate (FIG. 1, 407) of a heated plate impactor. Thus, for example RDX is vaporized 207 and the vaporized substances from the carrying particulate matrix on the plate are mixed 208 in gas phase with a flow containing reagent (which can be for example in an embodiment HNO3 vapor for a substance to be detected) the presence of the ions produced by an ionization source, embodied in an example the ionizing source 209 as an X-ray source to form adducts in form M+reagent (for example such as M+HNO₃ adducts), such adducts are formed. The sample is directed to the mass spectrometer to get finally analyzed by the mass spectrometer.

In FIG. 2 the mass spectrometer is an APITOF spectrometer, which is used in the detection 210. The mass related signal is detected, and processed 211 online for the mass results of the sample. The findings at the suitable masses representing the masses of substances in database having attributed as illicit defined are detected, and when threshold level for an alarm is observed, it triggers a decision algorithm to proceed according to the detection. For example, if the decision algorithm finds RDX or TATP, it is supposed generate an initiative signal that is controlling the conveyor belt and gate system to pick the piece of luggage, a suit case for example out of the line and guide it to the safety deposit box for further action or final disposal.

As the passenger that owns the luggage were known, by a taken photograph at the luggage entry and/or otherwise detected identity when leaving the luggage in, integration to such an identification system may trace the owner very fast and the security personnel can do the necessary actions.

FIG. 3 illustrates as a photograph an example of an embodiment of the system 300. The system is implemented in the figure as an arrangement. The FIG. 3 illustrates system modules as follows: 301 carrier flow suction blower, 302 sample extraction chamber, 303 virtual impactor, 304 conveyer belt, 305 an impactor/charger, 306 APITOF, 307 system control unit.

FIG. 4 illustrates a device according to an embodiment of the invention. The air jet pulses are produced by the means for air jet pulses production 401, comprising pressurized airline, for providing the carrier medium. The means 401 comprise also the valves to be controlled by the system for the timed pulses of the pressurized air, as embodied in the example to be 10 ms air pulses. The pulses are produced for detachment of particulate matter from the parcels and/or luggage surfaces. However, similar system can be used also for passengers, to detach particles from the clothing for example. The detachment can be performed in an extraction chamber 302, from which a sample flow is taken, for example at the rate of 4000 lpm. The blowers 402 and 403 are embodied in the example to provide air flows of 15 000 lpm and 11000 lpm, respectively. The number-8 symbol is used only for denoting to a blower rotor or similar part in the structure, but without intention to limit the structure only to the shown example.

The sample flow is directed to the virtual impactor (VI) 404, from which flow about 4000 lpm is taken out and the particles extracted to the 30.8 lpm minor flow are guided in flow line 414 to the heated plate impactor and charger system 405, as in this embodiment variant being integrated with the chemical ionization section 406 (104 in FIG. 1). The heated plate of the impactor is illustrated by the object 407. To the same chamber 0.1 sccm saturated reagent flow 409 is introduced to the chamber via a line to bring a reagent into the chamber 406, the reagent being comprised according to an embodiment of the invention, to get mixed to the sample flow (30 slpm for example). According to an embodiment the ions from/in the flows 411 to 412 can be guided by electric fields to increase the sampled ion concentration to the 412 flow.

The radiation source 408 is provided to produce X-ray radiation, to produce reagent comprising ions to the volume for recombination of the substances comprising ions to get them attached to the explosives, for example. Also direct photoionization can be used in suitable part in an ensemble of embodiments to produce ions.

Nitrate ions NO₃⁻ can be used in embodiments, also (HNO₃)ₙNO⁻ type substances in suitable part. A reagent can produce into the chamber positive and/or negative ions in a bipolar charging according to an embodiment of the invention. Also a reagent can be broken to produce ion-charge carrying fragments in suitable part.

The volumetric flow of the substance from the chamber is divided 410 so that 30 lpm is guided out via the corresponding flow line 411, but the sample flow 412 from it is guided to the line 412, leading to the API-TOF-spectrometer 413 for the mass analysis.

FIG. 5 illustrates a longitudinal cross section of device the device in FIG. 4 in applicable parts after the virtual impactor section. The sample is coming to the inlet 414 by a flow of 30.8 lpm, the heated plate impactor plate 407 collects the particles, and vaporize substances that are vaporizable at the plate temperature, (in an embodiment example, the plate temperature is below 400° C., but in another embodiment below 300° C., preferably below 250° C. in another embodiment, but around 200° C. in a preferred embodiment, but however over 100° C.). According to an embodiment the fine temperature setting is made according to a substance to be detected. The HNO3 agent as saturated substance is introduced to the chamber 406 via the line 409. The line 409 is indicated in the FIG. 5 at the middle of the longitudinal axis of the chamber. The HNO₃ is used, as an example to be mentioned, in ion production for the recombination purposes to combine the evaporated substances of interest to the radiation produced ions. According to an embodiment of the invention also other radiation produced ions can be used besides the NO₃⁻.

The X-rays are introduced for the ion production to the chamber of mixing and charging 406 via an x-ray window 508. Radial flow at the extraction part 411 is directed out, but the sample is going to the APITOF-pinhole 412 to the mass analysis.

FIG. 6A illustrates dependency of Reynolds number Re from the flow rate at 800 nm cut off size for the 2^{nd} stage of the virtual impactor, that is in the shown example embodied with 19 holes of 2.5 mm in diameter. At the 50% collection efficiency occurring at 800 nm, the flow was in the example 42 lpm per hole and the Reynolds number was 3000. The cutoff size dependence from the flow rate is illustrated in FIG. 6B.

FIGS. 7A and 7B illustrate dependency of Reynolds number Re from the flow rate for fixed nozzle size of 0.6 mm at 500 nm cut off size for the impactor that is in the shown example embodied with 30 holes. At the 50% collection efficiency occurring at 500 nm, the flow was in the example 1 lpm per hole and the Reynolds number was 2500. The cutoff size dependence from the flow rate is illustrated in FIG. 7B. Actual measurement data is indicated by dots.

FIGS. 8A, 8B illustrate graphics- and text-fields, respectively, on a virtual computer screen for an RDX measurement simulation data. In the screen the dependence of the signal as counts per second (cps) is illustrated as a function of the concentration. Zero limit and detection limit are indicated in the FIGS.

The RDX sample specs on screen are as follows: Integration time 12 sec, grey dots (in the middle of the confidence level bars): average of the measured data, Zero level: ˜1e-5, small RDX background from contaminated instrument, High resolution peak fitting used, Y-error bar height: 4*STD of measured data, X-error bar width: estimated from flow and temperature measurement, Colors: based on measurements, modelled probability that a given concentration is observed as a signal on the y axis. Detection limit: limit at which 95% of the signal data are above the highest 5% of the background, 12 sec detection limits: RDX: 70 ppq, PETN: 100 ppq, TNT: 2 ppt Could be still improved by cleaner instrument.

FIG. 9 illustrates example time series of an experiment, blank sample tested before each sample extraction. Sample extraction, and blank test alterations are indicated as a function of time for ion concentration. The peaks indicate extracted sample.

The impactor testings were performed with a feed in size selected particles, measure concentration up and downstream of the impaction region, obtain collection efficiency. Latex spheres in an atomized liquid were used as a suspension for test aerosol, which was made monodisperse with an electric classifier and diluted and dried before use for the impactor testing. The particle concentration was determined with a condensation nucleus based particle counter.

Also tests were made with evaporate TNT, RDX or PETN in a heat controlled flow, diluted to larger N2 flow, signal being measured with the CI-APITOF. By changing the dilution ratio to N₂ used in tests of the sample, the temperature and knowing the vapor pressure, the detection limits were obtained. It is also estimated that experiments done with the scientific inlet, a current embodiment version of the tested device, the inlet were probably more sensitive due to 10× higher TIC.

Impactor/charger were also tested with the following specs: Inlet flow rate 30 lpm, Impactor pressure drop 50 mbar, TIC˜400 000, Impactor plate temperature adjustable between 0-300 C, usually 200 C during experiments, Signal spikes 1-10 s. Sometimes was observed that background signal accumulates after tests. Background brought down by flushing the inlet with compressed air, approximately at rate of order of magnitude per night, so automated cleaning of the instrument can be made according to an embodiment of the invention during the non-duty periods by flushing the instrument by pressurized air feed. According to an embodiment the walls can be design to be continuously protected/flushed with flows at the walls with heated sheath air.

Mass sensitivity test with the device embodied for the RDX(/HMX) synthetized in-house to 3-w % solution of acetonitrile. By series of dilutions, acetone/RDX solutions of 0.001, 0.01, 0.1, 1, 10 ng/ul were prepared. Samples injected with a Hamilton microliter pipette to the heated impactor plate. Between each sample injection, a blank acetone sample was injected.

Analogies: 1×1×1 mm=1 ul=1 mg, 1 um particle=1e-15 l=1e-9 ul˜=1 pg, 1 ng=1e-12 l=1e-6 ul=one thousand 1 um particles.

The whole system tests, sample preparation was made. Results are indicated in FIGS. 10 to 19, as they were supposed to be shown on a screen reporting on the results.
2, 6 or 18 ul of 10 ng/ul acetone/RDX solutions first injected to a glass plate and let dry, resulting in masses of 20, 60 and 180 ng of RDX on the glass plate. Secondly, the glass plate was rubbed against a cardboard plate, resulting in something much less than 20, 60 and 180 ng on the cardboard plate
The cardboard plate was placed onto a plastic box, approximately the height of a luggage. The box was placed into the sample extraction chamber to various positions.
An air jet was used to detach particles from the cardboard plate, and the sample extraction chamber-virtual impactor-impactor-charger-APTIOF system sampled the detached particles.
Between each sample extraction from a cardboard plate, a blank sample from a clean cardboard plate was extracted
Sample extractions were also conducted directly in front of the virtual impactor and impactor to estimate the sample collection efficiency in the sampling line.

FIG. 1D illustrates system software module, form the view of operations of the system. Referring to the operations of the system as embodied in Examples 1 to 11, but especially in the example 11, an embodiment of the software module is disclosed. The box M illustrates a software module in a system, described as a Mediator or a mediation center. The mediation center is considered in an embodiment as acquiring data/information, sending information, sending triggering commands, observing timings, sending initiatives and observing their responses as well as keeping the system updated and utilizable for the user, so that the different modules can be operated under the system via the facilitated connections via the mediation center, if not directly from a module to another.

The reference numbers 101, 102, 103, 104, 105 and 106 denote to an interphase to the physical system elements, to communicate with them for controlling via sent command signals, to trigger/time actuators in the control of the mediation center M. The interphases can be two directional, so that measurement data can be acquired on the physical conditions, temperature, pressure, moisture, operating voltages, actuator status etc., to be logged for the controlling and maintaining the system in operation. The actuator and/or environmental as well as system elements heath signals are monitored. This is illustrated by the box illustrating measurement unit Mea. The measurement unit can also acquire the mass spectrometer signals and control the operation. The set up refers to the operation parameters that control the operation of the system. How the measurements and commands are made. The unit Decide comprises means to select, use and determine the decision making algorithms, to be applied to the operation maintenance.

The Communication part Com, in-ext.-remote comprises interfaces to the system internal communications between the actuators and software parts, (cited by the int) but according to an embodiment the communication part can also have communications interphases to external communications, for example to terminal devices outside the system. According to an embodiment of the invention the system operations can be controlled remotely, including also in an embodiment variant data acquisition control and/or measurement data transfer. Update is a module that concerns the measurement and control means so that the system has the latest version of the software available for the purposes defined in set up for the actuators and the communication.

Communication can utilize communication networks such as internet and/or cellular system, but is not limited only there to. The dashed line surrounding illustrates a closed relation of the modules comprising the interfaces to control mechanical Mech, electrical Elec and pneumatic Pneu operations, which are operable in suitable part in the modules 101 to 106. According to an embodiment these can be controlled by a unified module MEP.

According to an embodiment of the invention the Mediator M is controlling also the human interphase HI, the settings, security and/or displaying devices, as well as communication via the Com module.

The module Robo in an embodiment variant is reserved for a non-human, i.e. another external system operation, under of such in use, the system being remotely used according to the settings recorded and/or made in the mediator module M. Module Gr denotes to graphics, the settings about the displaying parameters, but also that what is shown and what is not shown. The Gr can operate also in suitable part with the other modules under the control of the Mediator M.

According to an embodiment the Mediator has also libraries (Lib as denoted) under the control, for chemical substances and their properties concerning ((Che Lib as denoted), but according to an embodiment also libraries for the physical properties (Ph Lib as denoted) of substances, their masses, mobilities, thermal parameters etc. to be used in the Mass spectrometer operation according to the type of the mass spectrometer.

The interface to pumps (Pump) and/or motors (Motor) are also under the control, so that fluids that are supposed to be transferred in the system are going from the container via the piping to the destination. Motors are operated to make for example a conveyor belt or sample exchanger to operate, to achieve mechanical translatory and/or rotational effects for the system operation.

### Example 1

In the following example of an embodiment and the related disclosed variants of it, the text describes the device and the method as well as tested performance of a prototype for rapid automated screening of luggage and parcels for explosives traces, used as an example, but applicably also to other illicit compounds detection, although the primary application in the example of the device in this example is intended use for screening of checked-in luggage at the airports. The device as a system element is disclosed being able to perform screening of up to 3000 units of baggage per hour. The device in the example performs the screening and detection of the explosives traces automatically. In case of positive identification, it informs security system of the customer by means of electronic communication so that the staff can make the further actions according to the reported information.

### Example 2

System tests are illustrated via virtual screen views presented in FIGS. 10 to 19. The virtual screen is demonstrated by the graphics and text fields surrounding rectangular line. The system was tested by preparing samples of 2, 6 or 18 μl of 10 ng/μl acetone/RDX solutions first injected to a glass plate and let dry, so resulting in masses of 20, 60 and 180 ng of RDX on the glass plate. Secondly, the glass plate was rubbed against a cardboard plate, consequently resulting in something much less than 20, 60 and 180 ng on the cardboard plate.

The cardboard plate was placed onto a plastic box, approximately the height of an average arbitrary luggage. The box was placed into the sample extraction chamber to various positions.

An air jet was used to detach particles from the cardboard plate, and the sample extraction chamber-virtual impactor-impactor-charger-apitof system sampled the detached particles.

Between each sample extraction from a cardboard plate, a blank sample from a clean cardboard plate was extracted

Sample extractions were also conducted directly in front of the virtual impactor and impactor to estimate the sample collection efficiency in the sampling line.

In some tests a cone was used in the test so that it was placed vertically to minimize gravitational losses of the extracted particles.

FIG. 10 illustrates a signal integral about 20 seconds to get enough signal for detection in the tests, although 2-3 s were sufficiently enough, the test results of an embodied system for samples that were denoted as of 20, 60, 180 ng as placed on the respective glass plate. The small circles are indicative of individual experiments; large circles mean values, blank data at 0 ng. According to the made tests, observed respective masses were as read from the screen about 9, near 20, and above 20 but less than 30 as shown in pgs as observed mass. Blank sample was slightly above zero in mass scale.

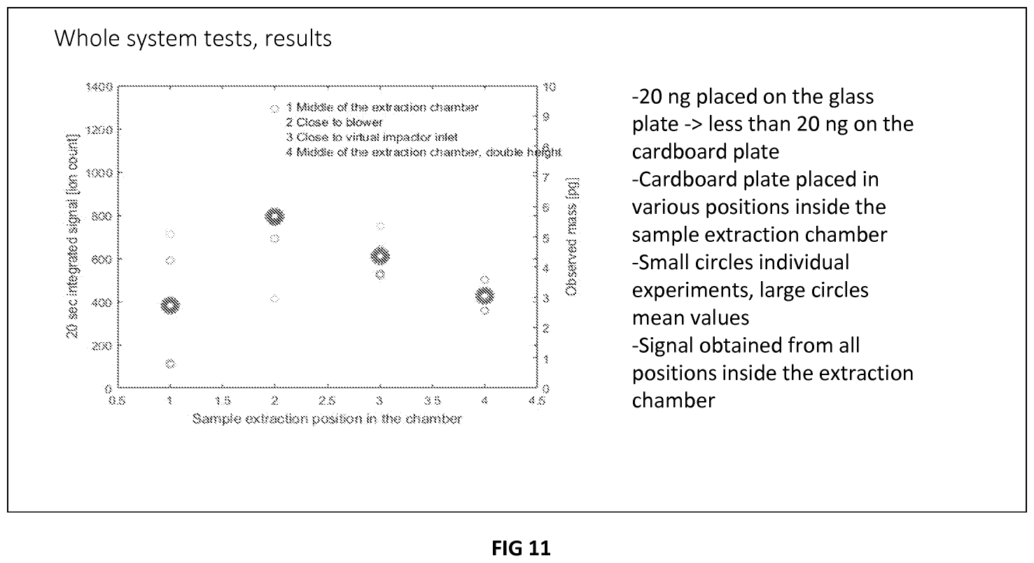

FIG. 11 illustrates a signal integral about 20 seconds, the test results of an embodied system for a sample of 20 ng, but the sampling made from various places of the system. The cardboard plate was placed in various positions inside the sample extraction chamber.

The positions were as follows: 1 Middle of the extraction chamber, 2 close to blower, 3 close to virtual impactor inlet, 4, middle of the extraction chamber, at a double height, as compared the first. Observed mass in pictograms varied from slightly below 3 to slightly below 6, at the first and second positions. Small circles individual experiments, large circles mean values. Signal obtained from all positions inside the extraction chamber.

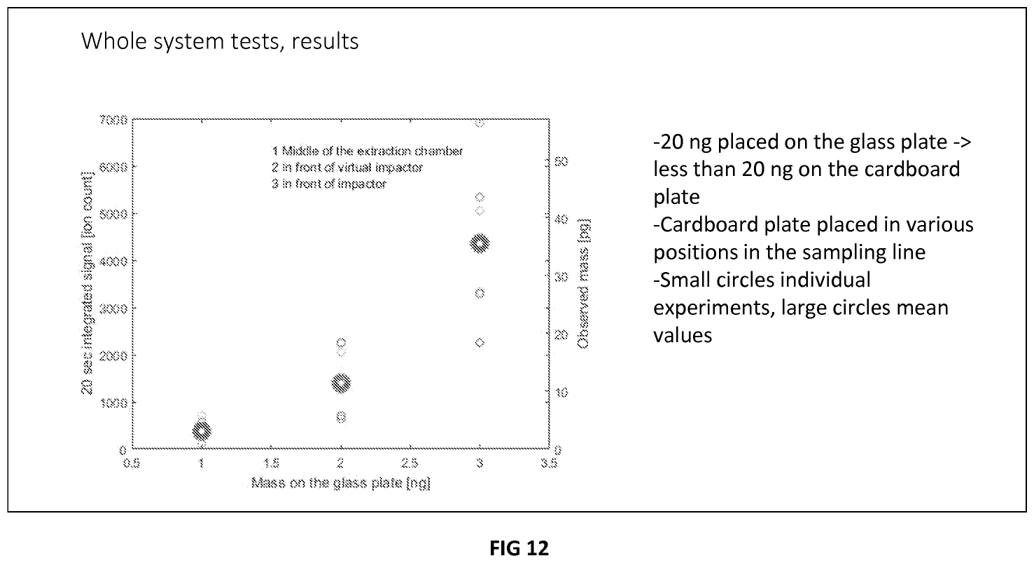

FIG. 12 illustrates a signal integral about 20 seconds for the cardboard plate placed in various positions in the sampling line, the test results of an embodied system for a sample of 20 ng. The cardboard plate positions were: 1 middle of the extraction chamber, 2 in front of virtual impactor, 3 in front of impactor.

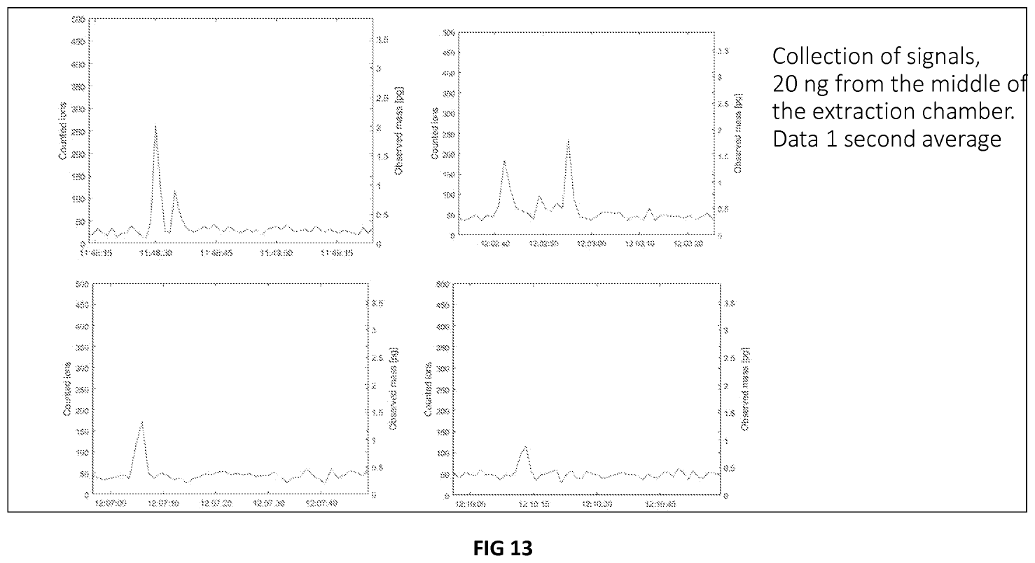

FIG. 13 illustrates collection of signals, 20 ng sample, the counts were taken when the sample was in the middle of the extraction chamber. Data has been represented as 1 second average.

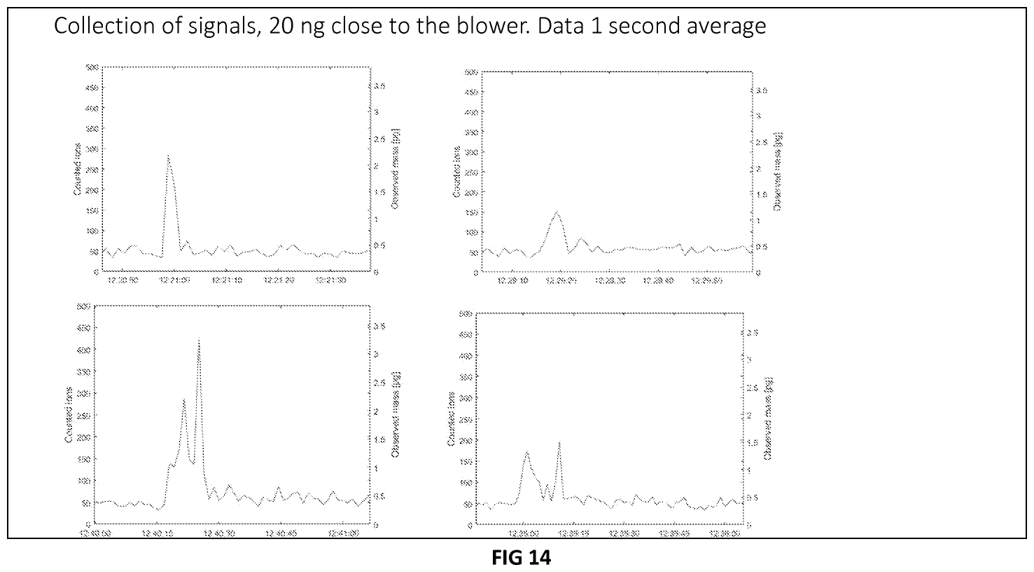

FIG. 14 illustrates collection of signals, 20 ng sample, the counts were taken when the sample was close to the blower. Data has been represented as 1 second average.

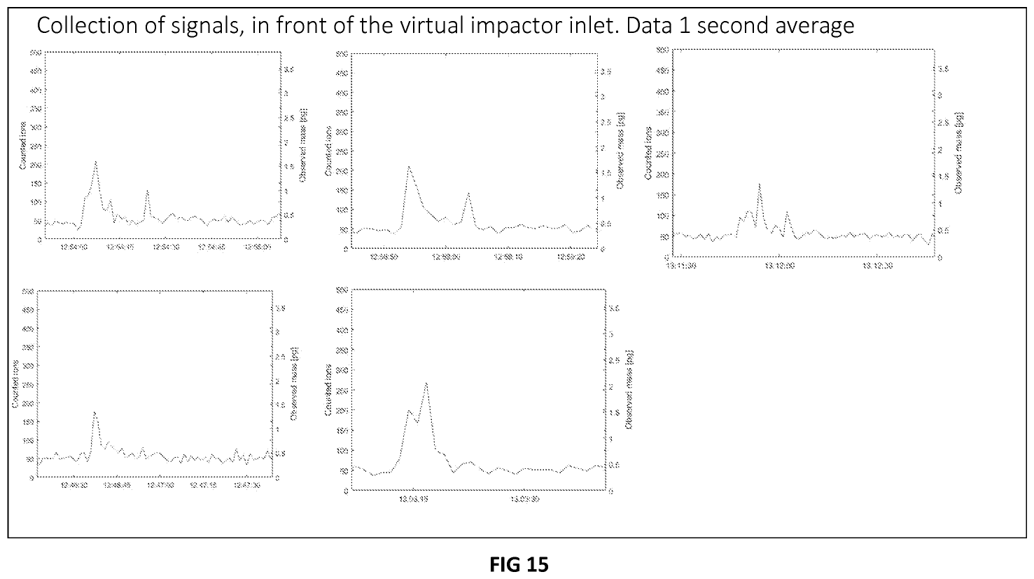

FIG. 15 illustrates collection of signals, 20 ng sample, the counts were taken when the sample was in front of the virtual impactor inlet. Data has been represented as 1 second average.

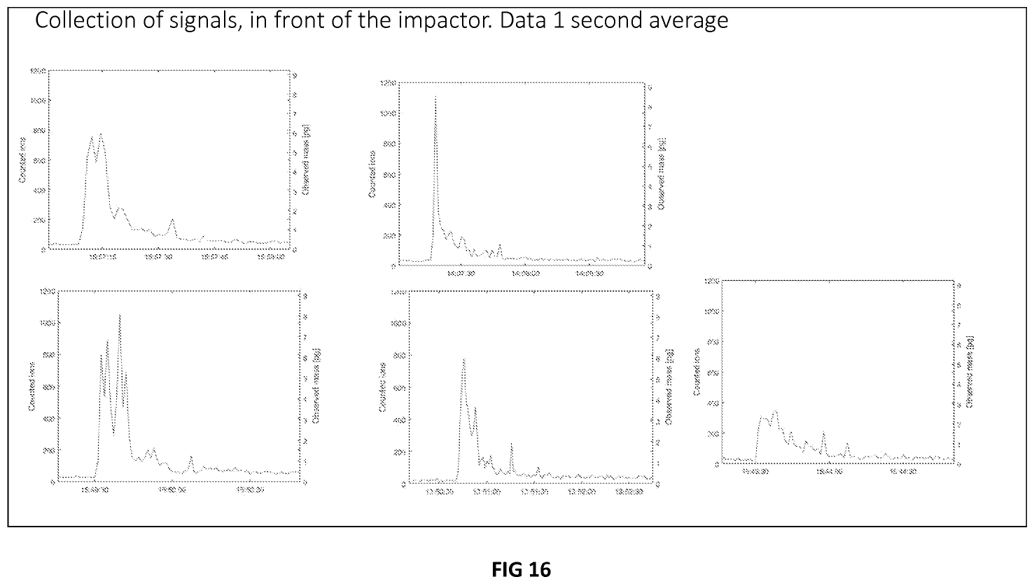

FIG. 16 illustrates collection of signals, 20 ng sample, the counts were taken when the sample was in front of the impactor. Data has been represented as 1 second average.

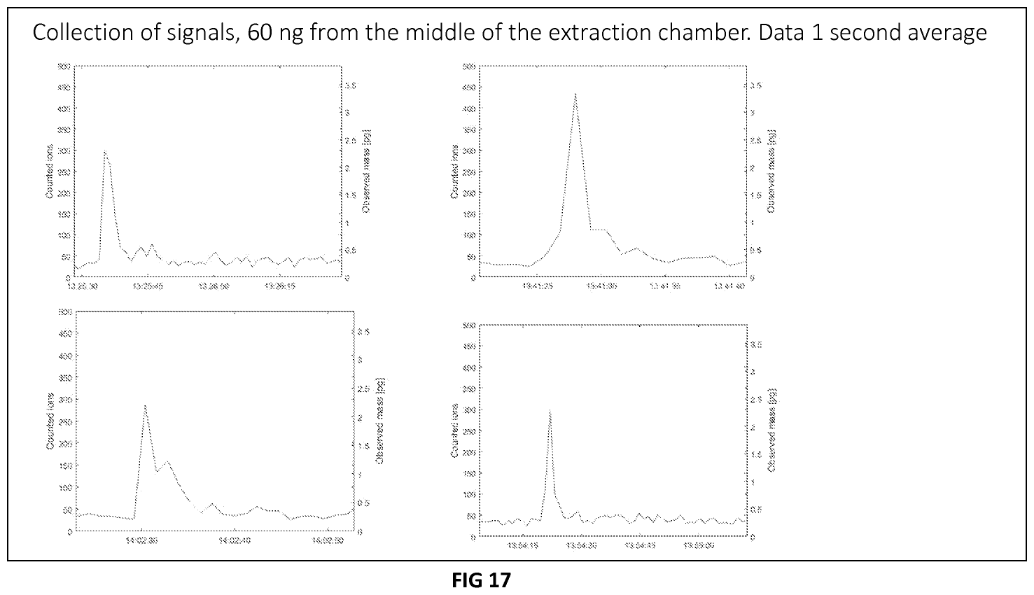

FIG. 17 illustrates collection of signals, 60 ng sample, the counts were taken from the middle of the extraction chamber. Data has been represented as 1 second average.

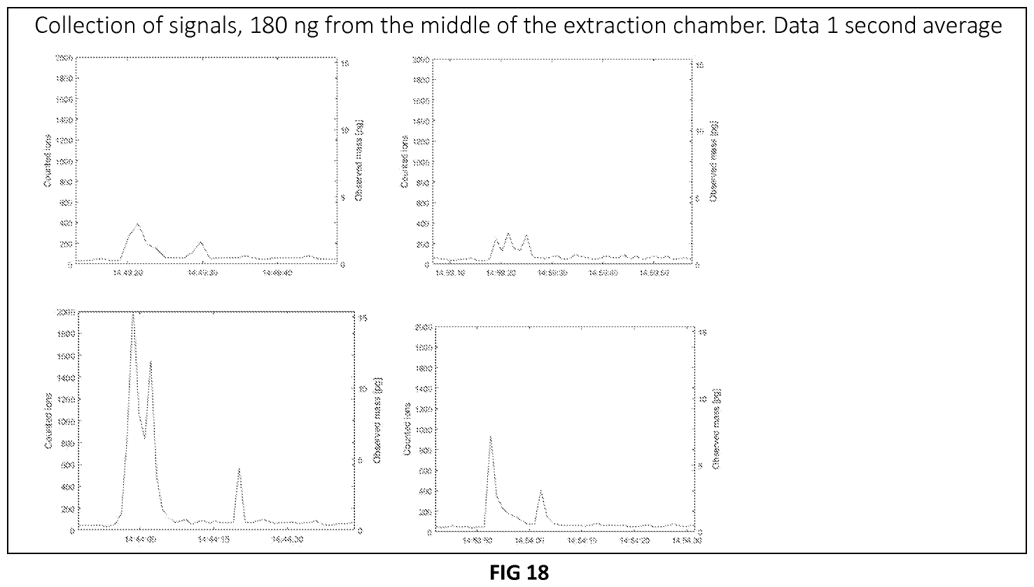

FIG. 18 illustrates collection of signals, 180 ng sample, the counts were taken from the middle of the extraction chamber. Data has been represented as 1 second average.

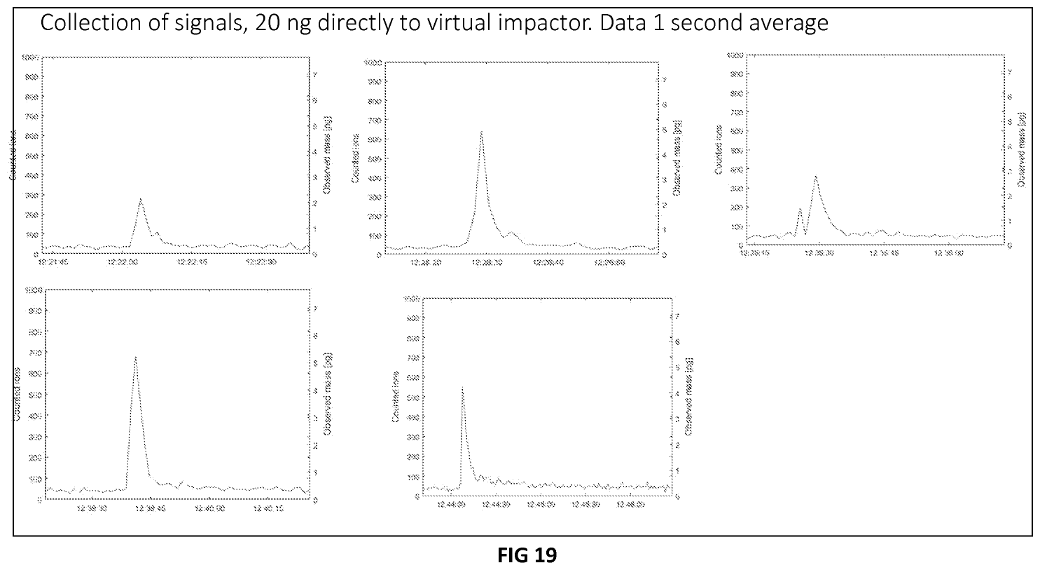

FIG. 19 illustrates collection of signals, 20 ng sample, the counts were taken directly to virtual impactor. Data has been represented as 1 second average.

Conclusively it was observed that
Instrument detects 3 pg of RDX from liquid solution
Instrument detects 20 ng of RDX detached from cardboard surface
Similar signal from 3 pg pipetting to impactor and 20 ng detachment test->ratio of 0.0001

About the extraction efficiency
Sample transfer efficiency from glass to cardboard ˜0.5
Sample extraction efficiency from cardboard with air jet ˜0.5
Sample extraction chamber sampling flow vs. carrier flow 0.33, measured
Virtual impactor 0.1, from optimum design
Particle bouncing from impactor, gravitational and inertial losses and other unknown losses 0.1-0.01
Total
->estimated efficiency close to the observed 0.0008-0.00008

However, impactor/charger inlet flow rate, HNO3 flow rate, and impactor plate temperature optimized based on preliminary results are not shown here.

### Example 3

The embodied system was used for illicit substances detection at a cargo gate. According to an embodiment of the invention the cargo gate in the example was a passenger luggage gate. However, according to an embodiment the cargo gate can be embodied for other type parcels, but according to an even further variant, a container interior can be analyzed by a portable system, which according to an embodiment is put on wheels for mobility to transport, and the system is provided with pressurized air source from bottles of inert gas, and a hoovering tubing connected to the virtual impactor, so that the container itself operates as the screening chamber. Similar way a truck, or its cargo volume as well as a deliver car's cargo volume, buss or similar can be monitored. Also a train or ship at least partly if not entirely, can be so examined.

According to an example, pieces of the luggage were exposed to the air jet pulses to release the surface attached particulate material. The detached particles became airborne and were sampled to the virtual impactor module, for pre-concentration, and then to the impactor plate, which was heated for vaporize substances that are vaporized at the impactor plate temperature. For nitrogen comprising explosives the plate was in the example set to 200° C. The vapors were led to the recombination and mixing chamber, where the nitrogen acid molecules combined to the explosives molecules by the radiation which was soft X-ray radiation generated by an X-ray tube operated at about 20 kV to 30 kV voltages. APITOF-analyzer was used to measure the mass spectrum for finding traces of explosives at the corresponding mass numbers.

### Example 4

At the example 3 recorded data was compared to a database comprising the mass numbers that match to the illicit substances in a profiles of such substances. Although the sample was revealed to be clean, clean from the explosives, the sample spectrum matched to a drugs profile, which made an alarm to the security personnel and the pieces of luggage was picked aside and was carefully inspected for the drugs.

### Example 5

At the example 3 recorded data was compared to a database comprising the mass numbers that match to the illicit substances in a profiles of such substances. Although the sample was revealed to be clean, it was far too clean to be a normal piece of luggage. This luggage piece did not carry the ordinary dust substances that were found from the average passenger's luggage surfaces. Thus, the spectrum was too clean to match to an average passenger's profile, and the profiling agent in the algorithm triggered an alarm to the automation system to take the piece of luggage out from the belt to be further inspected thoroughly. The security personnel found explosives and some traces of radioactivity that supported the illicit activities. The luggage was picked aside and the passenger was arrested from the entry to the plane.

### Example 6

At the example 3 recorded data was compared to a database comprising the mass numbers that match to the illicit substances in a profiles of such substances. Although the sample was revealed not to be clean, almost like normal pieces of luggage, but had a slight anomaly from the average spectrum and the profile of the local boarding. However, the passenger entering via a similar sampling port was treated in a similar way as the luggage, but very different spectrum was found, indicative that the passenger and the luggage were not from the same origin. Thus, the profiling agent in the algorithm triggered an alarm to the automation system to take the piece of luggage out from the belt to be further inspected thoroughly. The security personnel found nerve gases from the piece of luggage which was taken aside and the passenger was arrested.

### Example 7

A passenger had three suitcases, that entered to the example 3 disclosed inspection, to be made by the embodied system. The inspection was made, and one of the three suitcase specific profiles made the profiling agent in the algorithm suspicious and caused an extra sampling to be made with 10 s duration at a side line, to which the suspicious case was conducted by the automation driven belt and gate system. The suitcase profile with a longer sampling time revealed that the case was full of money, and the passenger was guided to the customs services to declare the origin of the money.

### Example 8

According to an example of an embodiment of the invention, profiles can be determined to luggage or other parcel or volume of example 3. According to an embodiment the profile can be made as a sub profile for a single piece of luggage for comparison to the other same luggage belonging items, and/or other profiles. This way the profiling agent algorithm can find and detect anomalies between the luggage and its belongings to reveal illicit content and/or origin.

According to an example of the invention, the profile of a passenger and/or his/her luggage can be saved. According to an embodiment of the invention such a saved profile can be compared to a previously recorded profile, and/or to that of other passenger profiles sharing the same departure/destination place. This way it is possible to monitor the development of the profile in time domain and consequently back ground information about the passenger and/or the luggage carried along.

For the purposes of profiling and its connection to other information of the passenger, it is not always necessary to record the whole mass spectrum that is available for comparison to be made by autocorrelation functions for example, but the masses to be monitored can be selected for different purposes to monitor. Thus, the system can monitor explosives at the known masses of the explosives molecules, drugs, nerve gases, note printing ink substances, radioactive substances, noble metals, etc. this kind of searching profiles can be applied to one target, but especially if any background monitoring gives a rise to suspicion for a more accurate sampling with a longer sampling time. The decision can be algorithm based, to be made according to a concentration and/or collected mass basis with thresholds to an ensemble of the observable quantities in the detection.

### Example 9

MTTD-ONE is a system product concept of automated Explosives Trace Detector for checked-in luggage screening. MTTD-ONE, as in follows also, for brevity, consists but not amounts to the following assembly units and subsystems as system elements: 1) Sampling tunnel, 2) Sample extraction system, 3) Sample concentrator, 4) Sample collection, vaporization and ionization unit, 5) Detector, 6) Software system module, and 7) Input-output devices.

According to an embodiment, in order to have high screening throughput of large objects such as luggage the air containing the possible extracted explosive traces are changed fast in the relatively large sample extraction volume, to achieve the exemplified 3000 items of luggage per hour.

The requirement of high sample flow also dilutes the sample, when the traces are detached from the luggage under observations. Thus, the detached traces of explosives in the sample, as airborne particulate form in room temperature, are concentrated to a pre-concentrator. According to an embodiment a mass spectrometer is used as a desirable instrument for the detection of explosives. This is because it can define species so accurately that the false positive detection becomes very unlikely if happens at all. On the other hand, mass spectrometers can get detected only charged ions or clusters, and that is why the pre-concentrated particles have to be vaporized and ionized before their detection.

### Example 10

A device was implemented for a checked in luggage screening. The system was described as follows:

### 1. The Sampling Tunnel

a. The tunnel to be installed over conveyor system for checked-in luggage. It was used as a chamber for sample extraction and the frame for peripheral devices. The tunnel was a semi-closed volume facilitating aerosol guidance from the target surface to the concentrator inlet, to provide to the detached airborne particles the passage to the pre-concentrator implemented by VI.

b. The tunnel might have built-in conveyor system integrated into device and integratable with conveyor system of the e.g. airport's baggage handling system.

According to an embodiment the sampling tunnel can be optionally a volume to be monitored, for example also a cargo volume of a vessel or ship.

### 2. The Sample Extraction System

a. Air jet system in an embodiment can be based on solid air pressure system with compressors and valves, preferably but not necessarily with a bottled air to gain independence from the solid line failure, or to have portability, for example on wheels.

Nozzles can be embodied as mounted on-to adaptable/moving handles or to the frame of the sampling tunnel. According to an embodiment variant, the pressurized air can be provided via own line of the system via piping for the purpose, especially in such an embodiment that is made portable or mobile on wheels. A robotic version in remote control can be also used when it is suspected that the sampling environment in a volume to be examined were too dangerous a human operator to be used in the sampling.

In such an embodiment the communication can be made via suitable wireless protocol, for example by using radio frequencies of cellular systems.

Bag detectors can be used in the automation of baggage handling and counting from the conveyor. Based on the signal from a bag detector the system control software commands a series of air pulses optimized to extract the possible explosive material from the surfaces of the bag. Compressor is of adequate power and duty cycle to provide enough compressed air for dislodgement system. Valves are large and fast enough to provide short pulses of high volume of compressed air.

A.a. to improve sample extraction additional devices such as acoustic cleaners or air blades might be utilized. These can be also used in such embodiment's implementations that are directed to inside-a-vessel-type operations in the sampling point control by a human operator inspecting a cargo volume, and/or robotic inspector.

b. When the particulate material to be sampled is released to the carrier flow, it carries the dislodged particles towards the detection system.

According to an example of an embodiment the flow can be generated with two large HVAC blowers, one for intake and one for exhaust. Other configurations are also possible. According to an embodiment, both the intake and exhaust flows are filtered using large HVAC filters. Within an example of an embodiment the carrier flow can be 15 m³ per minute, according to another example the carrier flow changes the air in a ˜2 m³ volume in time scale of ˜5 s time range.

c. Concentrator system entrances the particulate mass from the inflow of air at ca. 4000 liters per minute (lpm) with high capacity blower. The inflow might be varied according to the geometry and optimization of the sample extraction and transportation. In a portable device a blower can be incorporated to the mobile system.

FIG. 1A is referred for the virtual impactor operation. Main gas flow is from upper opening to the side openings. Lower opening has only a small flow. Heavy particles are drawn forward by their inertia whereas the majority of gas flow is drawn to sides. The FIG. 1B illustrates embodiment of the invention implemented by such a sample concentrator that has two virtual impactors in series.

### 3. Sample Concentrator

a. Extracted aerosol sample mixture is concentrated in regard to particles using a series of virtual impactors. Current flow ratio is 4000/800 lpm (stage 1) and 800/30 lpm (stage 2). Both the number of stages and the flow ratios are subject to change according to an embodiment variant in question.

A.b. According to an embodiment also a pre-concentrator optimized for ACSM-TOF might also be used in an optional embodiment of the invention, in supplement or addition.

b. In an embodiment a second stage is a circular array of 19 circular virtual impactors in parallel. The number of VI units, their shape and their arrangement are all subject to change according to an embodiment of the invention.

c. The sample concentrator can include a self-cleaning mechanism that removes dust and residual particulate matter from the system. Sheath air flows can be used to flush and protect the critical surface and/or other parts that may accumulate substances under interest because of temperature change related phenomena material that may pile to the corresponding locations to produce contamination like effects.

d. The sample concentrator can include a device cutting off particles larger than certain size from the sample flow such as a cyclone, a mesh, or an impactor. Even electrostatic precipitator that is clearly operated at the field charging regime can be used to remove large particles for an embodiment variant. The cut off device may also be heated to volatilize and sample the traces from large particles. Cut off device may include self-cleaning mechanism.

According to an embodiment of the invention rectangular orifices, slots were used in the virtual impactor, as an old and well known technique, as part of geometric design of the improved version of the VI. Sierra 235 (Hi-Vol) type geometry may be applicable in suitable part for the slots and/or stages as such, as applied to virtual impactor. The device had number of stages 5, 9 slots per stage, to yield 1130 lpm.

FIG. 1C is illustrating an operating principle of an impactor as such. Inertia of larger particles drives them on the impaction surface, which is heated in this case. This is further illustrated as connected to the system according to an embodiment of the invention in FIG. 5, so that the sample collection, vaporization and ionization unit design, but the X-ray source as such is omitted.

### 4. The Sample Collection, Vaporization and Ionization Unit

a. Multi-orifice (30) Impactor with Heated Impaction Plate was used in an embodiment of the invention. The parallel impactors collect particulate matter larger than 500 nm (the example value is not limiting the scope only to the shown example embodiment) on a heated plate which then vaporizes them into gaseous phase. The plate temperature is normally controlled in the range of 100-300 C depending on the optimization of vaporization and flows but can be ramped up to even higher temperatures for fast cleaning of the plate. The heating can be embodied by resistive based electrical heating, but in suitable part or optionally by an eddy current based magnetic heating.

a.b. According to an embodiment the whole unit is made self-cleaning. The walls can be heated from outside or a hot air stream could be introduced on the inner walls during the cleaning sequence in order to evaporate impurities. Pressure pulses can be introduced to remove the accumulated dust from the nozzles and narrowings and bigger airflow to blow the dust to a vent. Also automated “vacuum cleaner” head or cleaning air jets could be used for removing the dust. The spots mostly collecting the dust in the system can be made exchangeable or equipped with a mechanism facilitating easy or automated cleaning.

a.c. According to an embodiment multiple stage collection and evaporation can be used. The impactor might consist of several stages collecting and evaporating the particles with different cut-off sizes and thus reducing the possibility of clogging. Additionally heated filter media could be used for the collection and evaporation. Also in an embodiment variant the collecting substrate can be made exchangeable by a rollable film, stack of plates and/or plates on a ribbon fed from a belt for the respective embodiments.

b. According to an embodiment as reagent species, nitric acid vapor is introduced to the flow after sample vaporization. Other reagents or combinations of reagents are possible for negative or positive mode measurements.

c. Chemical Ionization (CI) Module with Soft X-Ray according to an embodiment is used. All of the sample passes through 4.9 kV soft x-ray cone as exemplified in the FIG. 1. Method using such charging generates both positive and negative ions which form clusters and/or reaction products with species in sample gas. The mixture is conducted to the detector instrument. Other ionization sources such as different x-ray, electron beam, radioactive sources or corona discharge can be used in respective embodiments.

### 5. Detector

a. API-TOF Mass Spectrometer in negative polarity can be used in an embodiment of the invention. API-TOF is operated in selected polarity. Whole sample mixture is induced to the mass spectrometer. Non-chosen polarity ions are however lost for a single line of one polarity. Mass spectra are measured at kHz-scale and summed internally according to an embodiment to spectrums representing few seconds of data.

b. CI-API-TOF with switching polarities or CI-TWIN API-TOF −/+ which can be used as in an embodied system, which is otherwise similar to single polarity measurements, but both negative and positive ions are measured.

c. ACSM TOF can be used in connection to the sampling made with the pre-concentrating section for the purpose, to replace entirely or partly a virtual impactor stage or stages. According to an embodiment of the invention the ACSM inlet would be then be where the heated impactor section is indicated in embodiments, in supplement or instead. ACSM TOF can be used according to an embodiment in supplement or instead of the CI-APITOF. According to an embodiment an extra stage of VI to concentrate the particles can be used, also for brining the flow from 30 lpm to 3 lpm for example.

d. In an embodiment of the invention in supplement or optionally other kind of mass spectrometers can be used as based and IMS detectors in suitable part.

### Example 11

### 6. Software System in Examples of an Ensemble of Embodiments

a. KRS_Cebro—analytical software, decision algorithms, controlling software commercially available can be used in suitable part. Also software libraries in suitable part can be used for code at least in part of such.
b. System control software to read the device sensors and control mechanisms (optical, temperature, valves etc.) excluding the mass spectrometer API-TOF. The key tasks for the system is to control the sample and reagent flows, the temperature of the impactor plate and the sample extraction connected to the bag detection. On top of that there is an additional measurements of temperature, relative humidity and pressure for monitoring the operation. The design is made to be flexible and expandable because the final operational concept as well as the measured variables can still change from an application and conditions specific way from one to another, and grow in number also due the different automated maintenance routines and operational may need for integration in varying environments.
   An embodied prototype software demonstration for study the system in operation was done with LabView which is a language developed for testing and control purposes as such. The embodied system control software is based on two parallel state machines which operate in different timescales. The core of the program is provided so that the data acquisition loop is syncing the state machine structure which sorts data for different purposes and makes decisions for further actions that can be easily changed and added without need to change the structure of the program. Actions can be also driven by value changes in the user interface such as setpoint changes or button switch. All analog data is read with 10 000 Hz rate which should be sufficient for most purposes. The usual data averaging interval is one second but shorter 100 ms interval is used for bag detection resulting still in averages of 1000 samples, thus low noise levels. Higher averaging rate is possible if needed for example for more accurate timing of the sample extraction. Resulting from above the main state machine has one second time for analyzing the data and making different actions based on that and user settings before the next data arrives. In this time scale the decision making algorithm can make the initiatives for the control and/or actions to be performed.
   The sample extraction control is in an embodiment at least partly separated to be tightly synced with the higher averaging rate of the bag detection data because lag time might cause inaccurate or even missed sampling Immediately when any of the bag detectors exceeds the given threshold value the program gives a series of counter driven digital pulses to control the magnetic valves behind the air pulses at the detachment of the particle. Optionally if needed some delay time between the detection of the bag and triggering of the pulses or dead time after the triggering can be added for respective embodiments. Also the rate, length and amount of the pulses in one series can be changed from the user interface. Typical parameters in testing have been series of 10 pulses with 10 Hz rate and 10 ms duty time each, but the length and rate as well as the number may be varied according to the system parameters. Additionally the sample extraction can be triggered also with a button from user interface which is a useful feature in testing. If the sample extraction is triggered automatically or manually there's digital signal sent out to be read for another instance. All the data as well as the operational settings and sample extraction events are saved in a file with 1 s time resolution.
c. Native TOF software, controlling spectra acquisition and parameters internal to mass spectrometer can be used in suitable part with the embodiments of the invention.
d. Libraries (specific files intended for detection of target substances) can be used for the detection, but also for the analysis. Especially in connection of profiling, the profiles also can be even recorded as to form a passenger or gate specific libraries.
e. Application specific GUI includes user interface intended for the use of security operators and service user interface for the use of service engineers.

1-13. (canceled)

14. A system for screening of traces of illicit substances, wherein the illicit substances comprise at least one of the following selected from the class of substances: toxics, explosives, super-poisons, nerve gases, narcotics, drugs, ELVOCs for illicit purposes and radioactive substances, the system comprising in the system as system element modules,
a spectrometry section (106),
an extraction section (105), (411), (412),
a sample extraction chamber (221), (302) for detachment of the illicit substances comprising materials, into a sample flow to be pre-concentrated in a virtual impactor,
a chemical ionization section (104),
a heated impactor section (103), with an impactor plate (133),
a pre-concentration module in a virtual impactor section (102), and
a sampling section (101),
wherein the sampling section is arranged to sample traces of the illicit substances (109) from the extracted material in extraction chamber (221), (302), into a high volume sample flow (402), to be carried to pre-concentration in the virtual impactor section (102) comprising at least one virtual impactor, to sample the flow-carried traces of the illicit substances, onto a heated impactor plate (133), (407), in series of the virtual impactor, the collected traces of the illicit substances (109) to be vaporized and led into the chemical ionization section (104), for forming ions in the ionization section (104), the ions produced therein, to be combined with the illicit substance selective reagent molecules that matches to the chemistry of the detectable traces from a reagent flow inlet (409), for forming aggregates with said illicit substances from the sample, the illicit substances having a substance specific mass, to be extracted (105) and the substance specific mass being analyzed in a mass spectrometer in the spectrometer section (106),
wherein the system comprises as system elements:
carrier flow suction blower (301), for the carrier flow,
sample extraction chamber (302), for extraction of sample,
at least a virtual impactor (303), in the virtual impactor section (102) for concentrating the sampled material to a smaller flow,
an impactor (133), (407) and charger (305), (405) for receiving the sample for charging (406), (408), wherein the impactor is a heated plate (133), (407) impactor arranged to be in the heated impactor section (103),
APITOF-unit (306), (413), for mass analysis of the sampled molecules,
the system control unit (307) to control the system.

15. The system of claim 14, wherein the system comprises as a system element a conveyor belt (304), for luggage transport.

16. The system of claim 14, wherein the reagent that matches to the chemistry of the detectable traces comprises at least one of the following: HNO₃, I₂, acetone ((CH3)₂CO), HCl and O₂.

17. A method of screening of traces of illicit substances with a system of claim 14, comprising
detaching illicit substances (109) comprising materials from an object to be screened,
sampling said detached air-borne materials into a sample flow (402),
pre-concentrating sampled air borne material in a virtual impactor (102),
collecting to concentrate the pre-concentrated air-borne onto a heated impactor plate (407),
heating the impactor plate (407) to thermally detach illicit substances,
leading the detached illicit substances to chemical ionization chamber (104), for ionization (108) and for forming aggregates with an illicit substance selective molecules,
analyzing molecules, including the illicit substances, by a mass spectrometer in a spectrometer section (106) of the system,
comparing the obtained masses to a mass-library of illicit substances,
reporting about the found illicit substances of the sample.

18. The method of claim 17, wherein the illicit substances comprise at least one of the following selected from the class of such substances: toxics, explosives, super-poisons, nerve gases, narcotics, drugs, ELVOCs for illicit purposes and radioactive substances and radioactive substances.

19. The method of claim 17, wherein the method comprises at the sample extraction chamber:
flapping (201), by air jet outlets connected to an extraction chamber,
detaching (202) particles (109) from luggage by air pulses,
transporting (203) the particles to the virtual impactor (102) in a carrier flow,
flushing (204) by changing air repeatedly in the extraction chamber.

20. The method of claim 17, wherein the method comprises concentration (102) of particulate mass from a high flow to a low flow:
concentrating (205) particles by a virtual impactor to an outlet flow
collecting (206) said particles from said outlet flow on a heated impactor plate.

21. The method of claim 17 wherein the method comprises for sample collection:
vaporizing (207) particles from the heated impactor plate (407),
mixing (208) vaporized gases in a gas phase with a reagent (HNO3),
ionizing (209) to form adducts with a reagent (HNO3) formed ions.

22. The method of claim 17, wherein the method comprises online detection and data analysis:
detecting (210) masses of adducts, of substances adducted with ions, with an APITOF
signal processing (211) online for the adduct mass,
utilizing (212) a decision making algorithm to make decision for the identity of the substance.

23. The method of claim 17, wherein the screening comprises screening of luggage from small to mid-size.

24. A device as a system element for screening of traces of illicit substances in a system of claim 14, comprising as system modules at least the following:
detachment module,
sampling module (101),
pre-concentrating module (102),
vaporizer module (103),
ionization module (104),
mass spectrometer module (106),
control module (M),
software code as a software module.

25. A system section or module as disclosed alone or in combination to an embodiment according to claim 24 for use in a device for luggage screening.

26. A non-transitory computer-readable medium on which is stored code for constitution of, for a system element module of system of claim 14,
control to the system, and/or
data acquisition to acquire data from said system.

27. The non-transitory computer-readable medium according to claim 26 which, when executed by a computer, performs the additional steps of:
detaching illicit substances (109) comprising materials from an object to be screened,
sampling said detached air-borne materials into a sample flow (402),
pre-concentrating sampled air borne material in a virtual impactor (102),
collecting to concentrate the pre-concentrated air-borne onto a heated impactor plate (407),
heating the impactor plate (407) to thermally detach illicit substances,
leading the detached illicit substances to chemical ionization chamber (104), for ionization (108) and for forming aggregates with an illicit substance selective molecules,
analyzing molecules, including the illicit substances, by a mass spectrometer in a spectrometer section (106) of the system,
comparing the obtained masses to a mass-library of illicit substances,
reporting about the found illicit substances of the sample.

28. The system of claim 15, wherein the reagent that matches to the chemistry of the detectable traces comprises at least one of the following: HNO₃, I₂, acetone ((CH3)₂CO), HCl and O₂.

29. The method of claim 18, wherein the method comprises at the sample extraction chamber:
flapping (201), by air jet outlets connected to an extraction chamber,
detaching (202) particles (109) from luggage by air pulses,
transporting (203) the particles to the virtual impactor (102) in a carrier flow,
flushing (204) by changing air repeatedly in the extraction chamber.

30. The method of claim 18, wherein the method comprises concentration (102) of particulate mass from a high flow to a low flow:
concentrating (205) particles by a virtual impactor to an outlet flow
collecting (206) said particles from said outlet flow on a heated impactor plate.

31. The method of claim 19, wherein the method comprises concentration (102) of particulate mass from a high flow to a low flow:
concentrating (205) particles by a virtual impactor to an outlet flow
collecting (206) said particles from said outlet flow on a heated impactor plate.

32. The method of claim 18 wherein the method comprises for sample collection:
vaporizing (207) particles from the heated impactor plate (407),
mixing (208) vaporized gases in a gas phase with a reagent (HNO3),
ionizing (209) to form adducts with a reagent (HNO3) formed ions.

33. The method of claim 19 wherein the method comprises for sample collection:
vaporizing (207) particles from the heated impactor plate (407),
mixing (208) vaporized gases in a gas phase with a reagent (HNO3),
ionizing (209) to form adducts with a reagent (HNO3) formed ions.
